(19) United States
(12) Patent Application Publication
(10) Pub. No.: US 2021/0330356 A1
(43) Pub. Date: Oct. 28, 2021

(54) DEVICE FOR FLUID TREATMENT OF THE SKIN

(71) Applicant: Image Derm, Inc., Los Angeles, CA (US)

(72) Inventors: Luis Del Rosario, Sun Valley, CA (US); Lee Del Rosario, Sun Valley, CA (US)

(73) Assignee: Image Derm, Inc., Los Angeles, CA (US)

(21) Appl. No.: 17/228,535

(22) Filed: Apr. 12, 2021

Related U.S. Application Data
(60) Provisional application No. 63/008,782, filed on Apr. 12, 2020.

(51) Int. Cl. A61B 17/54; A61M 1/00

(52) U.S. Cl. CPC A61B 17/545; A61B 2017/00761; A61M 1/774

(57) ABSTRACT

A skin-treatment system comprises a plurality of fluid reservoirs, a selection valve operatively connected to each of the plurality of fluid reservoirs, a first pump operatively connected to the selection valve, and a handpiece comprising a treatment tip configured to apply treatment fluid from one or more of the plurality of fluid reservoirs via the selection valve and the first pump to a skin surface. The treatment tip may comprise a vacuum inlet configured to aspirate waste fluid away from the skin surface. A momentary switch may be located on the handpiece. The momentary switch may have an “on” setting under which electrical current is allowed to flow to the first pump, and an “off” setting under which electrical current is prevented from flowing to the first pump. The first pump may be activated only when the momentary switch is in its “on” setting.

### CROSS-REFERENCE TO RELATED APPLICATIONS

This application claims the benefit of U.S. Provisional Application No. 63/008,782, filed on Apr. 12, 2020, which is hereby incorporated herein by reference in its entirety.

### BACKGROUND OF THE INVENTION

### 1. Field of the Invention

This invention relates to the field of treating the skin using fluid and/or abrasive particles.

### 2. Description of Related Art

Abrading the skin using a stream of abrasive particles is known as microderm abrasion. Treating the skin using a stream of skin treatment fluid, either alone or in conjunction with abrasive particles or abrasive surfaces, is also known. Examples of apparati and systems for treating the skin in such a manner include: U.S. Patent Publication No. 2016/0051436 by Del Rosario et al.; EPO Patent Publication No. 0324448(A1) by L.I.C.A. di ROSSO & C. S.n.c.; U.S. Pat. No. 6,139,554 to Karkar et al.; U.S. Pat. No. 6,432,113 to Parkin et al.; U.S. Patent Publication No. 2013/0158547 by David; and U.S. Patent Publication No. 2013/0066336 to Boone, I I I et al., all of which are hereby incorporated by reference.

### SUMMARY OF THE INVENTION

The present invention is of an apparatus and method for treating the skin using one or more fluids, optionally in conjunction with an abrasive surface.

According to an embodiment of the disclosed technology, a skin-treatment system comprises a plurality of fluid reservoirs, a selection valve operatively connected to each of the plurality of fluid reservoirs, a first pump operatively connected to the selection valve, and a handpiece comprising a treatment tip configured to apply treatment fluid from one or more of the plurality of fluid reservoirs via the selection valve and the first pump to a skin surface. The treatment tip may comprise a vacuum inlet configured to aspirate waste fluid away from the skin surface.

In one embodiment, the skin-treatment system further comprises a second pump operatively connected to the vacuum inlet of the treatment tip, a waste chamber operatively connected to the second pump and configured to receive the waste fluid, and a main power switch. The main power switch may have an “on” setting under which electrical current is allowed to flow to both the first pump and the second pump, and an “off” setting under which electrical current is prevented from flowing to both the first pump and the second pump.

In one embodiment, the skin-treatment system further comprises a momentary switch located on the handpiece. The momentary switch may have an “on” setting under which electrical current is allowed to flow to the first pump, and an “off” setting under which electrical current is prevented from flowing to the first pump. The first pump may be activated only when both the main power switch and the momentary switch are in their respective “on” settings.

In a particular embodiment, the plurality of fluid reservoirs includes two fluid reservoirs.

In another particular embodiment, each of the plurality of fluid reservoirs holds a treatment fluid. The treatment fluid may be different for each of the plurality of fluid reservoirs.

In yet another particular embodiment, the selection valve has a first setting that allows treatment fluid to flow from a first fluid reservoir of the plurality of fluid reservoirs, and a second setting that allows treatment fluid to flow from a second fluid reservoir of the plurality of fluid reservoirs.

In a further particular embodiment, the selection valve has a third setting that allows treatment fluid to flow from both the first fluid reservoir and the second fluid reservoir, resulting in a mixture of treatment fluids from both the first fluid reservoir and the second fluid reservoir being applied by the treatment tip. The selection valve may be configured to control of an amount of treatment fluid from the first fluid reservoir and an amount of treatment fluid from the second fluid reservoir, resulting in the mixture.

In one embodiment, the first pump is a positive-displacement pump.

The skin treatment system may further comprise a pinch valve operatively connected to the first pump. In one embodiment, the pinch valve is configured to inhibit treatment fluid from flowing through the first pump when the first pump is turned off.

In one embodiment, the handpiece further comprises a fluid control valve configured to control fluid flow to the treatment tip. The fluid control valve may be configured to control fluid flow within a range of fluid flow rates.

The skin-treatment system may further comprise a vacuum control valve operatively connected to the second pump. In one embodiment, the vacuum control valve is configured to control an amount of vacuum applied at the vacuum inlet of the treatment tip.

In one embodiment, an application end of the treatment tip comprises an abrasive feature, such as a diamond abrasive tip. In an alternative embodiment, an application end of the treatment tip is smooth.

In various embodiments, the momentary switch is in its “on” setting only when the momentary switch is being pressed. Treatment fluid is pumped from one or more of the plurality of fluid reservoirs via the selection valve and the first pump to the skin surface only when the momentary switch is being pressed. Treatment fluid is prevented from flowing from the plurality of fluid reservoirs via the selection valve and the first pump to the skin surface when the momentary switch is not being pressed.

Other features and aspects of the disclosed technology will become apparent from the following detailed description, taken in conjunction with the accompanying drawing, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following FIGURE. The drawing is provided for purposes of illustration only and merely depicts a typical or example embodiment of the disclosed technology. The drawing is provided to facilitate the reader's understanding of the disclosed technology and shall not be considered limiting of the breadth, scope, or applicability thereof. It should be noted that, for clarity and ease of illustration, the drawing is not necessarily made to scale.

FIG. 1 is a schematic diagram of an exemplary embodiment of the system of the present invention.

### DETAILED DESCRIPTION OF THE PREFERRED EMBODIMENTS

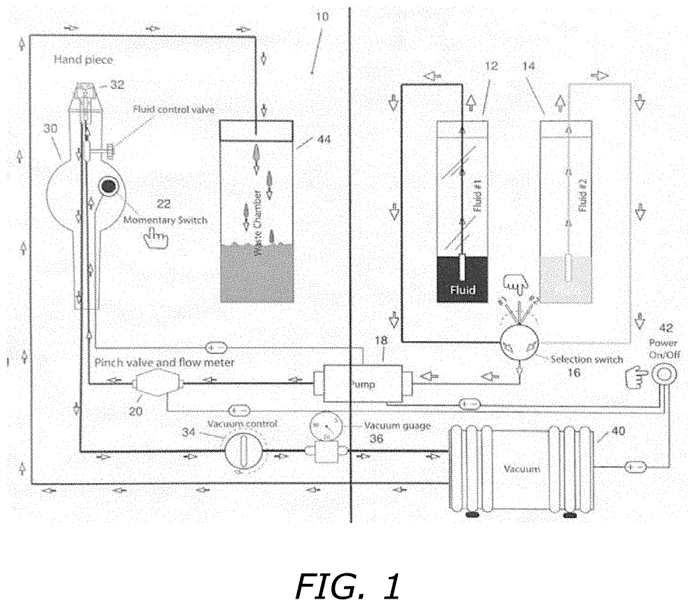

FIG. 1 is a schematic diagram of an exemplary embodiment of the system 10 of the present invention. In this embodiment two reservoirs 12, 14 hold different available fluids for treating the skin. The system can include more than two fluids and corresponding reservoirs. A valve 16 allows the operator to select which fluid treatment will be used, or what mixture of the two fluids will be used.

The system is turned on by activating power switch 42, which allows electrical power to flow to both positive pressure pump 18 and vacuum pump 40. The system can include a pinch valve 20 and a flow meter. Pinch valve 20 can include a solenoid and a mechanical pinch for pinching the flexible tube in which the fluid flows. The pinch valve presents fluid from being sucked through pump 18 when pump 18 is not turned on.

Pump 18 turns on when both the power switch 42 is in the on position, and when momentary switch 22 on hand piece 30 is being pressed. A fluid control valve 24 on hand piece 30 controls the flow of fluid to the treatment tip 32 of hand piece 30. Fluid control valve 24 can either control fluid flow within a range of fluid flow rates and can provide any degree of openness from all the way open to all the way closed. When pump 18 is on and fluid control valve 24 is open, fluid is pumped under positive pressure to treatment tip 32 where it is sprayed onto the skin being treated. After the fluid is sprayed out onto the skin being treated, the fluid is then aspirated away by the vacuum at the treatment tip provided by vacuum pump 40. The amount of vacuum at treatment tip 32 can be controlled using vacuum control 34 which may be a mechanical valve allowing a continuous range of vacuum to be provided at handpiece 32 including fully closed (no vacuum). The aspirated air/fluid mixture enters vacuum pump 40, is expelled from vacuum pump 40 at positive pressure, and is then collected in waste chamber 44.

The treatment tip 32 may be smooth, or may have abrasive features such as a diamond abrasive tip, such as are well known in the field, providing both a combination of dermal abrasion and fluid treatment simultaneously.

Momentary switch 22 may be configured such that it is activated by the operator lightly squeezing handpiece 32 using one or more fingers or his entire hand, allowing the operator to turn on and off the fluid flow without taking his eyes off the treatment area and without requiring any additional motions by the operator. By providing a momentary switch on handpiece 30 to easily turn on and off delivery of treatment fluid, the system provides various advantages over prior system in which vacuum was used to both withdraw fluid from one or more treatment reservoirs and deliver it to the treatment tip, and simultaneously aspirate the spent fluid, including that the present system provides more flexibility and control in the turning on and off, and flow rate delivery, than such prior systems, and greater ease of use. Being able to easily and quickly control the flow rate delivery is important at least because the fluids used in such dermal treatment systems are typically very expensive. Additionally, an operator may wish to temporarily turn off the flow of fluid while the operator uses an abrasive end of the treatment tip for abrasive purposes. Once the operator is finished with abrasive-only operation, the operator can turn on flow of the treatment fluid once more.

Although the present invention has thus been described in detail with regard to the preferred embodiments, it should be apparent to those skilled in the art that various adaptations and modifications of the present invention may be accomplished without departing from the spirit and the scope of the invention. Accordingly, it is to be understood that the detailed description and the accompanying drawing as set forth hereinabove are not intended to limit the breadth of the present invention, which should be inferred only from the following claims and their appropriately construed legal equivalents.

What is claimed is:

1. A skin-treatment system comprising:
a plurality of fluid reservoirs;
a selection valve operatively connected to each of the plurality of fluid reservoirs;
a first pump operatively connected to the selection valve;
a handpiece comprising a treatment tip configured to apply treatment fluid from one or more of the plurality of fluid reservoirs via the selection valve and the first pump to a skin surface, the treatment tip comprising a vacuum inlet configured to aspirate waste fluid away from the skin surface;
a second pump operatively connected to the vacuum inlet of the treatment tip;
a waste chamber operatively connected to the second pump and configured to receive the waste fluid;
a main power switch having
an “on” setting under which electrical current is allowed to flow to both the first pump and the second pump, and
an “off” setting under which electrical current is prevented from flowing to both the first pump and the second pump; and
a momentary switch located on the handpiece, the momentary switch having
an “on” setting under which electrical current is allowed to flow to the first pump, and
an “off” setting under which electrical current is prevented from flowing to the first pump;
wherein the first pump is activated only when both the main power switch and the momentary switch are in their respective “on” settings.

2. The skin-treatment system of claim 1, wherein the plurality of fluid reservoirs includes two fluid reservoirs.

3. The skin-treatment system of claim 1, wherein:
each of the plurality of fluid reservoirs holds a treatment fluid; and
the treatment fluid is different for each of the plurality of fluid reservoirs.

4. The skin-treatment system of claim 1, wherein the selection valve has:
a first setting that allows treatment fluid to flow from a first fluid reservoir of the plurality of fluid reservoirs; and
a second setting that allows treatment fluid to flow from a second fluid reservoir of the plurality of fluid reservoirs.

5. The skin-treatment system of claim 4, wherein the selection valve has a third setting that allows treatment fluid to flow from both the first fluid reservoir and the second fluid reservoir, resulting in a mixture of treatment fluids from both the first fluid reservoir and the second fluid reservoir being applied by the treatment tip.

6. The skin-treatment system of claim 5, wherein the selection valve is configured to control of an amount of treatment fluid from the first fluid reservoir and an amount of treatment fluid from the second fluid reservoir, resulting in the mixture.

7. The skin-treatment system of claim 1, wherein the first pump is a positive-displacement pump.

8. The skin-treatment system of claim 1, further comprising a pinch valve operatively connected to the first pump.

9. The skin-treatment system of claim 8, wherein the pinch valve is configured to inhibit treatment fluid from flowing through the first pump when the first pump is turned off.

10. The skin-treatment system of claim 1, wherein the handpiece further comprises a fluid control valve configured to control fluid flow to the treatment tip.

11. The skin-treatment system of claim 10, wherein the fluid control valve is configured to control fluid flow within a range of fluid flow rates.

12. The skin-treatment system of claim 1, further comprising a vacuum control valve operatively connected to the second pump.

13. The skin-treatment system of claim 12, wherein the vacuum control valve is configured to control an amount of vacuum applied at the vacuum inlet of the treatment tip.

14. The skin-treatment system of claim 1, wherein an application end of the treatment tip is smooth.

15. The skin-treatment system of claim 1, wherein an application end of the treatment tip comprises an abrasive feature.

16. The skin-treatment system of claim 15, wherein the abrasive feature is a diamond abrasive tip.

17. The skin-treatment system of claim 1, wherein the momentary switch is in its “on” setting only when the momentary switch is being pressed.

18. The skin-treatment system of claim 17, wherein treatment fluid is pumped from one or more of the plurality of fluid reservoirs via the selection valve and the first pump to the skin surface only when the momentary switch is being pressed.

19. The skin-treatment system of claim 18, wherein treatment fluid is prevented from flowing from the plurality of fluid reservoirs via the selection valve and the first pump to the skin surface when the momentary switch is not being pressed.

20. A skin-treatment system comprising:
a plurality of fluid reservoirs;
a selection valve operatively connected to each of the plurality of fluid reservoirs, the selection valve having:
a first setting that allows treatment fluid to flow from a first fluid reservoir of the plurality of fluid reservoirs, and
a second setting that allows treatment fluid to flow from a second fluid reservoir of the plurality of fluid reservoirs;
a first pump operatively connected to the selection valve;
a pinch valve operatively connected to the first pump and configured to inhibit treatment fluid from flowing through the first pump when the first pump is turned off;
a handpiece comprising
a treatment tip configured to apply treatment fluid from one or more of the plurality of fluid reservoirs via the selection valve and the first pump to a skin surface, the treatment tip comprising a vacuum inlet configured to aspirate waste fluid away from the skin surface, and
a fluid control valve configured to control fluid flow to the treatment tip within a range of fluid flow rates;
a second pump operatively connected to the vacuum inlet of the treatment tip;
a vacuum control valve operatively connected to the second pump and configured to control an amount of vacuum applied at the vacuum inlet of the treatment tip;
a waste chamber operatively connected to the second pump and configured to receive the waste fluid;
a main power switch having
an “on” setting under which electrical current is allowed to flow to both the first pump and the second pump, and
an “off” setting under which electrical current is prevented from flowing to both the first pump and the second pump; and
a momentary switch located on the handpiece, the momentary switch having
an “on” setting under which electrical current is allowed to flow to the first pump, and
an “off” setting under which electrical current is prevented from flowing to the first pump;
wherein the first pump is activated only when both the main power switch and the momentary switch are in their respective “on” settings;
wherein the momentary switch is in its “on” setting only when the momentary switch is being pressed;
wherein treatment fluid is pumped from one or more of the plurality of fluid reservoirs via the selection valve and the first pump to the skin surface only when the momentary switch is being pressed; and
wherein treatment fluid is prevented from flowing from the plurality of fluid reservoirs via the selection valve and the first pump to the skin surface when the momentary switch is not being pressed.
